(19) United States
(12) Patent Application Publication
(10) Pub. No.: US 2007/0092970 A1
(43) Pub. Date: Apr. 26, 2007

(54) High-throughput screening assay for Na, K-ATPase using atomic absorption spectroscopy

(76) Inventors: Dong Cuan Liang, Vancouver (CA); Sikander Singh Gill, Vancouver (CA); Rajwant Kaur Gill, Vancouver (CA)

Correspondence Address: Dong C. Liang, 1001 E Pender Street, Vancouver, BC, VGA IWZ, CA

(21) Appl. No.: 11/585,261

(22) Filed: Oct. 24, 2006

Related U.S. Application Data
(60) Provisional application No. 60/729,716, filed on Oct. 25, 2005.

(51) Int. Cl. G01N 33/48

(52) U.S. Cl. 436/63

(57) ABSTRACT

A method of chemical analysis involving Flame Atomic Absorption Spectroscopy (FAAS) and Graphite Furnace Atomic Absorption Spectroscopy (GFAAS) in combination with flux assays to directly measure intracellular or extracellular ion concentration to analyze Na⁺,K⁺-ATPase activity.

### RELATED APPLICATIONS

This application claims the benefit of prior filed provisional, Application No. 60/729,716 dated Oct. 25, 2005.

### BACKGROUND OF THE INVENTION

Na⁺, K⁺-ATPase is a pump in the membrane of cells. It is a highly-conserved integral membrane protein that is expressed in virtually all cells of higher organisms. This pump is responsible for the maintenance of ionic concentration gradients across the cell membrane by pumping 3 Na⁺ out of the cell and 2 K⁺ into the cell. Since this pump requires the expenditure of energy by hydrolysis of ATP for this action, it is, therefore, called as Na⁺,K⁺-ATPase. It has been estimated that roughly 25% of all cytoplasmic ATP is hydrolyzed by sodium pumps in resting humans. In nerve cells, approximately 70% of the ATP is consumed to drive Na⁺,K⁺-ATPase.

The catalytic subunit of the Na⁺—K⁺-ATPase is expressed in various isoforms (α₁, α₂, α₃) that are detectable by specific antibodies. The functional enzyme is comprised of an alpha and beta subunits; families of isoforms for both subunits exist. Na⁺,K⁺-ATPase is one of the other members of the family of cation pumps. The other prominent members of this family include gastric H⁺,K⁺-ATPase, sarcoplasmic and endoplasmic reticulum Ca²⁺-ATPase, plasma membrane Ca²⁺-ATPase, and plasma membrane H⁺-ATPase of fungi and higher plants, as well as heavy metal pumps. The ionic transport conducted by Na⁺,K⁺-ATPase creates both an electrical and chemical gradient across the plasma membrane. This is critical for both the cell and for directional fluid and electrolyte movement across epithelial sheets for example:
The membrane potential of the cell at rest is a manifestation of the electrical gradient which is the basis for excitability in nerve and muscle cells.
Export of sodium from the cell provides the driving force for several facilitated transporters, which import glucose, amino acids and other nutrients into the cell.
Translocation of sodium from one side of an epithelium to the other side creates an osmotic gradient that drives absorption of water.

Depending on cell type, there are between 800,000 and 30 million Na⁺,K⁺-ATPases on the surface of cells. Their distribution on the cell surface may be fairly even, or clustered in certain membrane domains.

Abnormalities in the number or function of Na⁺—K⁺-ATPases are thought to be involved in several pathologic states, particular heart disease and hypertension. Well-studied examples include:
Several types of heart failure are associated with significant reductions in myocardial concentration of Na⁺—K⁺-ATPase.
Excessive renal reabsorption of sodium due to oversecretion of aldosterone has been associated with hypertension in humans.
The down regulation of Na⁺,K⁺-ATPase is responsible for lung clearance in alveolar tissues which is corrected with upregulation of Na⁺,K⁺-ATPase)

The malfunctioning of this pump due to anoxic conditions by the loss of ATP or inhibition of this pump with drugs like cardiac glycosides such as Digitalis, or mutations in the pump lead to accumulation of Na⁺ and depletion of K⁺ within the cells. This causes depolarization of the resting membrane potential.

Human Na⁺—K⁺-ATPase, is emerging as an important drug target ever since cardioglycosides such as digoxin and digitoxin began to be used for the treatment of congestive heart failure and related conditions. Similarly, Protein Kinase C (PKC) induced phosphorylation of cardiac alpha-1 Na⁺—K⁺-ATPase is a likely target for treatment with antihypertensive compounds that have PKC-inhibitory activity. Additionally, other types of ATPases like gastric H⁺—K⁺-ATPase, the sarcoplasmic reticulum Ca²⁺-ATPase, the plasma membrane Ca²⁺-ATPase, and the Neurospora H⁺-ATPase are attractive pharmaceutical targets.

For the purpose of finding suitable therapeutic modulators, activity of Na⁺—K⁺-ATPase has been determined by both non-cell based and cell based assays. In the former category, the activity of Na⁺—K⁺-ATPase has been determined by ATP hydrolysis using purified preparation of the enzyme; the activity was calculated by measuring the difference between the ATP hydrolysis observed in the presence and absence of ouabain.

The cell based assays on the other hand are based on several distinct techniques such as electrophysiology, fluorescence, radio-ligand binding and radio-tracer flux assays. The electrogenic characteristics of the ATPase have been measured by voltage-clamp techniques in several cell types. In smooth muscle cells, the physiological roles of Na⁺—K⁺-ATPase have been examined mainly by measuring ion flux, isometric tension, and membrane potential with blockers for Na⁺—K⁺-ATPase such as ouabain, and incubation with K⁺-free solution. The patch-clamp technique would be of advantage as intra- and extracellular ionic conditions can be controlled and undesired ionic currents could be eliminated by various blockers during the experiment.

With the fluorescence method, the Na⁺—K⁺-ATPase flow measurements have been carried using the Na⁺-sensitive and electrochromic dye named fluorescence label 5-iodoacetamidofluorescein and the styryl dye RH 421.

Radiotracers based Rubidium⁸⁶ (Rb⁸⁶) uptake is another tool for determining the functional characteristics of Na⁺—K⁺-ATPase. Radio-ligand binding assays using ³H-ouabain binding has also been employed to study Na⁺—K⁺-ATPase. Similarly, Rb⁺ influxes via Na⁺—K⁺-ATPase and ouabain-resistant pathways have been studied in human blood lymphocytes. The radiotracer determination of Na⁺—K⁺-ATPase activity by Rb⁸⁶ has advantages in throughput, miniaturization and the signal-to-noise ratio. The radioactive ⁸⁶Rb⁺ efflux assay, for example, is a relatively unsafe and inconvenient technique in that the radioactive isotopes required are harmful to human operators, the half-life of the isotopes restricts the time duration of experiments, and there are radioactive waste disposal considerations to be dealt with.

These issues can be resolved by using non-radioactive Rb⁺ that can be substituted for K⁺ as a tracer in bioassays. Based on this tracer, K⁺-channel activity has been reliably determined with Rb⁺ using atomic absorption spectrometry (AAS) in different cell types. Therefore, in the present study, we developed an assay for examining Na⁺—K⁺-ATPase in cultured cells of CHO-K1, HEK-293 and L(t)K cell lines.

However, current technologies for screening pump activity, such as fluorescent indicators, radiotracer-based ⁸⁶Rb uptake, and radioligand binding assays pose such problems as high background noise, half-life problems, quenching effects and pH sensitivity. Moreover, all of the techniques described above are an indirect measure of intracellular ion concentration. Accordingly it is an object of this invention to provide a method for preparing and analyzing sample cell cultures for ion channel activity such that a direct and accurate measurement of intracellular ion concentration may be achieved.

Therefore, the need exists for the invention of a high throughput screening (HTS) assay that will effectively and rapidly screen Na⁺,K⁺-ATPase activity.

### SUMMARY OF THE INVENTION

The present invention pertains to experimental methodologies for biopharmaceutical research, particularly for the analysis of drug candidates for therapeutic effects on Na⁺,K⁺-ATPase. The invention describes a method of preparing sample cell cultures for analysis, and using a unique flux (in take of ions) assay and the techniques of flame atomic absorption spectrometry (FAAS) or graphite furnace atomic absorption spectrometry (GFAAS) to directly measure the intracellular ion content of those cell cultures, enabling the measurement of ion flux and ion channel activity.

An advantage of this invention is that the experimental methodology described herein provides a high throughput way for scientists to accurately determine the therapeutic effects of novel candidate compounds for screening against Na⁺,K⁺-ATPase target.

### BRIEF DESCRIPTION OF DRAWINGS

Additional features and advantages of the invention will be disclosed in the following detailed description, given by way of example, of a preferred embodiment taken in conjunction with the accompanying drawings, wherein:

FIG. 1 is a block diagram of the procedure for preparing sample cell cultures.

### DETAILED DESCRIPTION OF THE INVENTION

There are two methods to determine the activity of the compounds against this target. The first method of preparing the cell culture samples for analysis is called the Tracer Ion Method. The second method for preparing cell culture samples for analysis is called the Direct Measure Method. The method of this invention enables the measurement of ion flux through cell membrane Na⁺,K⁺-ATPase to provide information on Na⁺,K⁺-ATPase activity. The method described below is directed to the analysis of Na⁺,K⁺-ATPase, however, it will be readily appreciated by those skilled in the art that the present invention may be adapted to analyze the activity of other pumps (e.g. H⁺,K⁺-ATPase)

Tracer Ion Method

The rubidium ion and lithium ions are similar to the potassium ion and sodium ion, respectively, both in terms of physical and chemical properties such as molecular size and ionic charge. In view of the tracer ions, sodium or potassium may be called native ions. Because of these similarities, these ions are able to pass through many respective channels and transporters, with varying permeability coefficients. An advantage of using rubidium or lithium as tracer ions is that they are not present in biological systems. When the tracer ion concentration of the cell sample is eventually measured after experimental manipulation, the background signal will be very small. In the tracer ion method, while incubating cell cultures expressing Na⁺,K⁺-ATPase in the extracellular solution containing a tracer ion rubidium, either with or without addition of a compound (i.e. a drug being tested), Na⁺,K⁺-ATPase will pass the rubidium tracer ions into the cell. In an alternative protocol, lithium ions would be passed out of the cell by Na⁺,K⁺-ATPase (if the cells are preloaded with lithium) because Na⁺,K⁺-ATPase passes 3 sodium ion (tracer lithium ions) out of the cell in exchange of passing two potassium ions (tracer rubidium ions) into the cell. Measurement of the concentration of the tracer ion provides a measure of Na⁺,K⁺-ATPase activity. The addition of the compound enables one to identify molecules, which modulate Na⁺,K⁺-ATPase activity, hence isolating potential future drug candidates targeting Na⁺,K⁺-ATPase. The compound being referred to is the actual drug candidate that the researcher is studying. Due to the low background signal associated with the Tracer Ion Method, the measurement of ion channel activity can be more sensitive than the Direct Measure Method.

Direct Measure Method

In Direct Measure Method, the cell cultures are incubated with potassium ions instead of tracer ions. Therefore, when the ion concentration is eventually measured, it will be a direct measurement of potassium ion movement into the cell through the Na⁺,K⁺-ATPase activity. Conversely, efflux of sodium ion from the cells Na⁺,K⁺-ATPase activity can also be carried. Similarly, activity of Na⁺,K⁺-ATPase can be monitored by determining sodium ions in the sodium free extracellular solution as the sodium ions are passed out of the cell in exchange of potassium or tracer rubidium ion present in the extracellular solution. This method may have the advantage that it is more accurate than the tracer ion method because sodium or potassium ion movement through the Na⁺,K⁺-ATPase may be measured directly. But the results may suffer from high background noise.

The direct measure method details a complete protocol for measuring the sodium or potassium ion content of cell cultures where cells are incubated with a compound in the presence of potassium ions while Na⁺,K⁺-ATPase are active.

The method and procedures below apply equally to the Direct Method and Tracer Method. The only difference is that wherever KCl(potassium chloride) is specified to be used in the Direct Measure Method, RbCl would be specified to be used in the Tracer Ion Method.

Sample Preparation

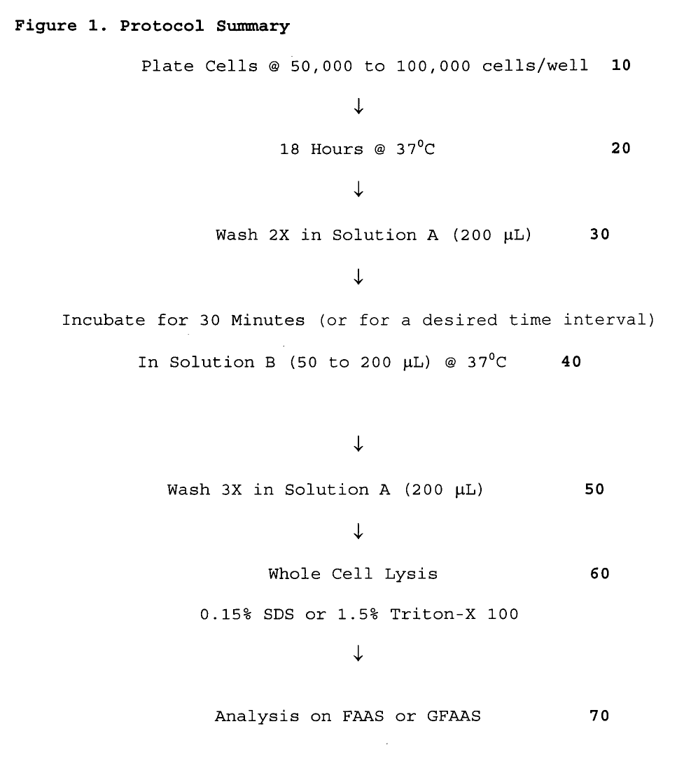

Referring to FIG. 1, an outline of the method described below is shown.

a. Tissue Culture

The cells used for the analysis can be from any desired cell source expressing Na⁺,K⁺-ATPase and having any other characteristic of interest. This methodology can be used for any type of ATPase pumps. This assay was developed with three endogenously expressing Na⁺,K⁺-ATPase cell lines (human, Chinese hamster, and another of mouse origin). Common cell lines including Chinese Hamster Ovary, Human Embryonic Kidney or fibroblast cell lines may also be used.

The cells naturally (endogenously) express Na⁺,K⁺-ATPase to detect sufficient activity or the Na⁺,K⁺-ATPase may be over expressed due to tumorgenic transformations, or they can be expressed as a result of transfection with gene(s) encoding protein(s) of the Na⁺,K⁺-ATPase in the appropriate expression vehicle (stable or transient transfection).

The cells to be used are incubated and cultured by traditional means (which are well known to those skilled in the arts). The cells are then removed from the culture vessel with trypsin solution, and diluted to a final stock concentration of 50,000-200,000 cells/mL 10. Trypsin is a digestive enzyme that is used to dissolve the bonds between cells and the culture vessel and among the cells, thus allowing the cells to be physically removed and manipulated. Cells are then plated out; 200 μL of the trypsinized cells are seeded into each of the 96 wells of a 12×8 well format microplate. This provides a density of 50,000-100,000 cells/well. The multiwell plate may be biocoated or electrostatic surface treated for cell adherence to the surface. The cells are then allowed to incubate at 37° C. for a typical incubation period of 18 hours 20. The exact incubation period used in an experiment will depend on the desired final cell density, the cell line used, and on the ion channel expression. The purpose of the incubation period is to allow the cells to grow, express ion channels, multiply to increase the cell density in the microplate wells and allow cells to adhere to the surface of the microplate wells.

b. Assay

The cells are washed twice with 200 μL of a wash medium solution, called Solution A. Solution A provides the cells with an isotonic environment and functions to wash the cells before addition of the tracer rubidium ion in Solution B. The liquid handling steps can be performed manually or automatically by modern robotics. In either case, the washing technique involve the use of a micropipette with a fine tip. The pipette tip must be carefully inserted into the sample well, and carefully draw up the Solution without drawing up any cells or damaging them in the process.

Solution A consists of 15 mM HEPES, 140 mM Sodium Chloride (NaCl), 5.4 mM Potassium Chloride (KCl), 1 mM Magnesium Chloride (MgCl₂), 0.8 mM Sodium dihydrogen phosphate (NaH₂PO₄), 2 mM Calcium Chloride (CaCl₂), and 5 mM Glucose, pH 7.4 set with Sodium hydroxide (NaOH). Solution B is the same as Solution A with the replacement of sodium chloride with Choline Chloride or potassium chloride is replaced with rubidium chloride, the tracer ion salt (depending on whether the Direct or the Tracer Ion Method is being used.

All chemicals and biological substances described and used by this invention are commercially available. HEPES is an acronym for 4-(2-hydroxyethyl)piperazine-1-ethanesulfonic acid. Its chemical formula is C₈H₁₈N₂O₄S. Choline is a natural amine that is found in body tissue. In Solution A, the KCl, MgCl₂ and NaH₂PO₄, and CaCl₂ mineral salts are required to create a balanced isotonic environment. Glucose is required as a nutritional supplement for the incubating cells. A compound (i.e. the drug being tested) may be added to the Solution B to determine if there is an effect on Na⁺,K⁺-ATPase.

When using this protocol to determine functional compound interaction with Na⁺,K⁺-ATPase, 50-200 μL of the following mixture in Solution B is added; Solution B and compound. The effect of the agonist used in the methodology of this invention is to inhibit or stimulate Na⁺,K⁺-ATPase activity. The entire microplate is incubated at room temperature or at 37° C. for a period of time. This period of time is an experimental factor, and can vary from just a few minutes to 3 hours. After the incubation period, cells are washed three times with 200 μL of Solution A.

The cells are then lysed with 0.15% Sodium Dodecyl Sulphate (SDS) or 1.5% Triton-X 100 or 300 mM HCl or 160 mM HNO₃ or any other non-ionic detergent. The SDS and Triton-X 100, are common lysing agents, that are ionic or non-ionic detergent, respectively. These are readily available and sold commercially. These agents lyse the cells by solubilizing the lipid bi-layer of the cell membranes. The resulting lysate (the homogenous liquid mixture of dissolved cellular components) is then suitable for immediate analysis, either by FAAS or GFAAS.

C. FAAS/GFAAS

Regardless of which method is used to determine the Na⁺,K⁺-ATPase activity (i.e. the Direct Measure Method or the Tracer Ion Method), there are two different spectroscopic techniques that can be used to actually measure the ion concentration: FAAS or GFAAS. The major difference between the two techniques is that GFAAS is more sensitive than FAAS. When the concentration of ions in the sample is expected to be high, and/or there is a large enough volume of sample, FAAS should be used.

Alternatively, when the ion concentration is expected to be very low, and/or there is not much volume of sample to work with, GFAAS would be more appropriate as GFAAS is capable of receiving a smaller sample size than FAAS and has a higher sensitivity than FAAS. The detection level of Rb⁺ using the ICR 8000 (Aurora Biomed Inc.) is highly sensitive, within the range of 0.05 ppm to 5 ppm.

d. Data Processing

The method described here can be used to determine whether a compound is a blocker of the Na⁺,K⁺-ATPase, a non-blocker (no effect), or an activator (inducing Na⁺,K⁺-ATPase activation). Such test compound can be a potential cancer drug being tested for side effects on Na⁺,K⁺-ATPase, or a potential drug specifically designed to target Na⁺,K⁺-ATPase. For example, if it were found that addition of a compound resulted in a lower concentration of potassium or tracer ions in the cell sample than without the addition of the compound, then this would indicate that the compound is a blocker of the Na⁺,K⁺-ATPase (that is, the compound inhibited the influx of potassium or rubidium ions into the cells). Similarly, if an addition of a compound resulted in a lower concentration of sodium or tracer lithium ion in the extracellular sample than without the addition of the compound, then this would indicate that the compound is a blocker of the Na⁺,K⁺-ATPase and it reduced the efflux of sodium or lithium ions out of the cells.

Alternatively, if it were found that the addition of a compound resulted in a higher concentration of potassium or rubidium ions in the cell sample than the sample without the addition of the compound, then this would indicate that the compound is a stimulator of the Na⁺,K⁺-ATPase (that is, the compound increased the influx of potassium or rubidium ions into the cells). Similarly, if the addition of a compound resulted in a higher concentration of sodium or lithium ions in the extracellular sample than the sample without the addition of the compound, then this would indicate that the compound is a stimulator of the Na⁺,K⁺-ATPase (that is, the compound increased the efflux of sodium or lithium ions out of the cells).

If the addition of a compound resulted in no more or no less potassium or rubidium (alternatively sodium or lithium) tracer ions in the sample than in the sample without the addition of the compound, then this would indicate that the compound is a non-blocker of the Na⁺,K⁺-ATPase, or neutral (that is, the compound had no effect on the flow of these ions into or out of the cells).

Controls (Table 1)

In most instances where the method of this invention is practiced, controls will also have to be done in order to make the results meaningful. The controls generally follow the method of the invention, with the following differences described below. Generally, for Control #1 through #4, after cells are washed two times in Solution A, 50-200 μL of Solution B is added to the sample wells with tracer element, with and without compound. Cells are incubated in Solution B for a set period of time, washed three times in Solution A and lysed as described in FIG. 1. Table 1 is a description of Controls #1 through #4 with contents of Solution B.

Control #1 gives the experimenter information on the concentration of the ions present in the cell before the cells are influenced by the assay. The purpose of Control #2 is to show the activity of the Na⁺,K⁺-ATPase in the native ion (eg. rubidium tracer ion or sodium ion) free medium, with the compound present. Here, it is recommended that a standard positive and negative control compound be tested. For example, Ouabain is known to block Na⁺,K⁺-ATPase and would be an ideal negative control compound whereas (ω-Agatoxin IV is known to block calcium channels and would be ideal as a positive control compound. This will give the experimenter information on the movement of ions without addition of ions (eg. potassium or tracer) in the presence of a compound which blocks the Na⁺,K⁺-ATPase and in the presence of a compound which does not block the Na⁺, K⁺-ATPase.

The Control samples include, but are not limited to, the following:

(a). a negative control indicating the normal Rb⁺ uptake in the

(b). absence of any Drug; and

(c). a positive control indicating the Rb⁺ uptake in a medium containing a known blocker.

The purpose of Control #3 is to show the activity of the Na⁺,K⁺-ATPase in a medium containing native ion or tracer ions, without any influence from the drugs. Control #3 gives the level of basal flux under experimental conditions.

The information obtained in Control #4 will identify effects of a compound under the conditions of the assay and can be compared with Control #1 and #6. This control will also determine if the experimental environment affects compound activity (when known compound is used). The analysis of ion movement in Control #4 will determine if the compound is inhibiting ion flow through Na⁺,K⁺-ATPase by the addition of a compound known to block Na⁺,K⁺-ATPase activity.

TABLE 1
Description of Controls with Contents of Solution B.

| | Potassium or | | |
|---|---|---|---|
| | Tracer Ion | | |
| Control # | or Choline* | Compound | Information |
| 1 | X | X | Ion concentration |
| | | | present in cell line |
| | | | before manipulation. |
| 2 | ✓ | X | Movement of added |
| | | | ions, indicating |
| | | | basal flux. |
| 3 | X | ✓ | Identifies compound |
| | | | influence on Na⁺, K⁺- |
| | | | ATPase when compared |
| | | | to Control #1. |
| 4 | ✓ | ✓ | Method of screening |
| | | | for Na⁺, K⁺-ATPase |
| | | | inhibitors. |

*Addition of potassium or rubidium, depending on which method used, Direct Measure Method or Tracer Ion Method. In the alternative protocol addition of choline is carried to measure sodium efflux or lithium efflux.

What we claim as our invention is:

1. A method of measuring ion flux through a cell membrane Na⁺,K⁺-ATPase pump, comprising:
a) washing cells expressing said Na⁺,K⁺-ATPase in a first isotonic solution that does not contain the tracer ion passing through said Na⁺,K⁺-ATPase pump;
b) incubating said cells in a second isotonic solution containing a tracer ion capable of passing through said ion channel;
c) washing said cells in said first isotonic solution, so as to create a liquid mixture containing said cells and having an extracellular concentration of said ion that is approximately zero;
d) lysing said cells so as to create a homogenous liquid mixture; and
e) measuring a concentration of said ion in said homogenous liquid mixture using one of: Flame Atomic Absorption Spectroscopy and Graphite Furnace Atomic Absorption Spectroscopy.

2. The method of claim 1, wherein cells were loaded with lithium tracer ion that pass out of the cell through Na⁺,K⁺-ATPase into the second isotonic solution.

3. The method of claim 1, wherein said second isotonic solution includes a compound operative to modulate activity of said Na⁺,K⁺-ATPase pump.

4. The method of claim 1, wherein said second isotonic solution includes potassium chloride or rubidium chloride.

5. The method of claim 1, wherein said first isotonic solution includes choline chloride or sodium chloride.

6. The method of claim 1, wherein said second isotonic solution includes mineral salts from a group consisting of KCl, MgCl₂, NaH₂PO₄ and CaCl₂.

7. The method of claim 1, wherein said first isotonic solution includes mineral salts from a group consisting of KCl, MgCl₂, NaH₂PO₄, and CaCl₂.

8. The method of claim 1, wherein said second isotonic solution includes glucose.

9. The method of claim 1, wherein said first isotonic solution includes glucose.

10. The method of claim 1, wherein said second isotonic solution includes HEPES.

11. The method of claim 1, wherein said first isotonic solution includes HEPES.

12. The method of claim 1, wherein said cells are lysed using 0.15% SDS or 1.5% Triton-X 100.

13. The method of claim 1, wherein said channel is a Na⁺,K⁺-ATPase pump.

14. The method of claim 13, wherein said ion is sodium.

15. The method of claim 13, wherein said ion is lithium.

16. The method of claim 13, wherein said ion is rubidium.

17. The method of claim 13, wherein said ion is potassium.

18. The method of claim 13, wherein said cells are from Chinese hamster.

19. The method of claim 13, wherein said cells are from human origin.

20. The method of claim 13, wherein said cells are from rat origin.

21. The method of claim 13, wherein said cells are from mouse origin.
